(19) United States
(12) Patent Application Publication
(10) Pub. No.: US 2014/0107499 A1
(43) Pub. Date: Apr. 17, 2014

(54) Medical Information Management System

(71) Applicant: True Process, Inc., Glendale, WI (US)

(72) Inventors: Todd Dunsirn, Shorewood, WI (US); Doug Frede, Mequon, WI (US); Charles Downey, Milwaukee, WI (US)

(21) Appl. No.: 13/836,767

(22) Filed: Mar. 15, 2013

Related U.S. Application Data
(60) Provisional application No. 61/713,216, filed on Oct. 12, 2012.

(51) Int. Cl. A61B 5/00; A61M 5/172

(52) U.S. Cl. CPC A61B 5/0022; A61M 5/172
USPC 600/483; 604/151

(57) ABSTRACT

A medical device communication system is described. The system includes a plurality of medical devices, a message listener programmed to wirelessly receive a plurality of messages from the plurality of medical devices, a storage device in communication with the message listener for storing the messages, a message distribution device for delivering the messages to a plurality of locations including a system database, an analytics database, and an integration engine. The system is configured such that the system database performs extract, transform, and load operations to generate system specific message objects configured to include the received message used to generate the system specific message object, the analytics database computes and displays a real time analytic information stored in each system specific message objects, and the integration engine is programmed to transmit the system specific message objects and receive a plurality of feedback from a central information system.

### CROSS REFERENCE TO RELATED APPLICATIONS

This application claims the benefit of U.S. Provisional Application No. 61/713,216, filed Oct. 12, 2012, hereby incorporated by reference in its entirety.

### BACKGROUND OF THE INVENTION

The present invention is related to the field of communication (retrieval and sending of data) and storage of medical information commonly received from medical devices in the hospital environment. Conventional technology requires that separate servers and databases be employed for storage of information received from each type of device, or each manufacturer of a device. For example, a heart monitor may be equipped to output the sensed signal which may be sent to a server and stored into a database. A medical drug pump may also be configured to output a signal regarding dosage levels supplied to a patient. Conventional technology does not allow the output from the drug pump and the heart monitor to be commonly stored and commonly readable in the same database. Different manufacturers may program their own devices with proprietary software that requires specialized databases in order for the output to be readable and properly archived in a database. Currently, health care facilities may employ several different proprietary servers and databases for all the various types and brands of machinery commonly used in a hospital environment.

### SUMMARY AND OBJECTS OF THE INVENTION

By way of summary, the present invention is directed to the management of medical device and medical related information. An effect of the present invention is to collect information from a multitude of medical devices and sources on a common network and make it available to qualified individuals. A primary object of the invention is to provide an apparatus that may collect, send, archive, and categorize information from any device. An additional object of the invention is to have a single data management system that is capable of receiving information transmitted using a wired or wireless connection (hereinafter referred to as wireless for convenience) from any device used in the medical field and allow qualified users to access that information.

The medical information management system includes a central database programmed to send and receive data to and from a plurality of medical devices. The medical devices may include, but are not limited to, medical diagnostic equipment, medical pumps, medical imaging devices, and any device capable of gathering information from a hospital patient.

The medical devices may be programmed with software allowing them to wirelessly input data to the central database. Configuring the medical devices to communicate wirelessly with the central database allows, for example, ambulances to be equipped with medical diagnostic and treatment devices that communicate with the central database over a wireless network.

The central database may be located anywhere and accessible on a server via the internet. Accordingly, the central database may be implemented within “the cloud,” meaning that computing servers and/or data storage capacity is provided on demand using a large number of distributed systems. The cloud allows users to avoid the cost of a huge datacenter while simultaneously allowing for rapid growth if needed. The cloud may be specifically configured by a medical company for use with its product or the cloud may be any generic storage space that is accessible via the internet. Having a cloud contain the central database further facilitates access to the underlying data since it enables remote access with any device equipped with telematic software and hardware. The telematic equipment may be retrofitted of into any existing medical device or may be built into the device during manufacturing.

The term telematics is known to include devices that utilize telecommunication technology, as well as the internet, for the purpose of transmitting and storing information. Telematics may also include allowing wireless, remote control of an object using telecommunication and internet technology. Additionally, telematics may include any technology used to communicate such as satellite or radio communication.

A handshake device programmed in the central server and database translates all received data into a language that is readable and interchangeable by a multitude of devices. One example of such a device may include a nurse's station or electronic health record. The handshake device allows one, common software to be used to track and compartmentalize all received data to the central server. This prevents situations requiring multiple databases and multiple software programs to read, track, and store all the different information received by the nurse's station. The same principle may be applied to the entire hospital's information infrastructure, allowing a seamless integration of all information that is gathered from medical devices. Delivery of the information may be facilitated using a subscription service that allows certain users and systems to subscribe to the information of interest.

A filtering device in communication with the central database may be used as well. The filtering device may include software programmed on the central database, or anywhere else, and programmed to filter the data into various groups of distinguished categories. This allows the data to be easily stored and to link related data together. The filtering device prevents a qualified person from having to sift through data to search for a single piece of information. For example, if the amount of morphine administered over the course of a month is desired, the filter device may be used to gather and store all data relating to the quantity of morphine administered based on a common identifier such as the patient name, room or bed id, but is not limited to these types of identifiers. For reasons such as this, the filtering device may be programmed as the administration sees fit, and custom tailored to the specific needs of the organization. It does not matter what device or what manufacturer supplies the information. The handshake device and filtering device may make the data all commonly readable by a single device including a personal computer, a smart phone, and a tablet computer or any other related device.

The server and database may also include data mining applications configured to allow users and/or the server to gather information from other devices associated with a patient that may be related to a particular event, such as the delivery of morphine described above. The data mining applications allow users to easily determine the effect that the morphine delivery had on, for example, heart rate, blood pressure, SPO2, and other vitals.

One example of a medical device that may benefit from this technology includes a medical liquid pump. Medical liquid pumps are commonly used in the medical field to administer liquid medication, nutrition, blood, etc. The pump may be manufactured or retrofitted with telematic software and hardware to allow it to communicate with the central database. A message listener device may be programmed to wirelessly receive messages from any number of medical pumps in operation. These pumps may be inside ambulances, in emergency rooms, in patient rooms, or anywhere. A message distribution device may include a message queue to store messages and a message broker to allow the hospital to deliver the messages following receipt and processing by the medical information management system as described in detail below, that are received by the central database to a plurality of locations including an operational database like the hospital's main system database, an analytics database, and an integration engine. The system database may be programmed to extract data from outside sources, transform it to fit operational needs (which can include quality levels), and load it into the end target (database, more specifically, operational data store, data mart or data warehouse). This process is commonly referred to as ETL in the industry. Reports may then be generated following the ETL stage.

The analytics database may compute and display real time analytic information using an analytics engine. An integration engine may be programmed to transmit the messages and receive a plurality of feedback from a central information system through the use of a specific integration partner plug in which allows communication with Cerner, Epic, or other databases. The central information system may include a regional system that is responsible for many health care facilities or any other overseeing entity such as Cerner or Epic. The hospital's main information system database may then be programmed to produce orders for pumps anywhere in the field. An order broker implemented by the medical information management system may then transmit the orders wirelessly to each pump. The orders may include a drug library with a plurality of drugs identified as well as dosage information for a particular patient. A file distribution broker programmed on the central database assigns the proper pump with its specific order. A health care provider may then receive the order wirelessly on the pump as it displays the order with the necessary drug's information.

The pumps may further each include a locator allowing the central database and the hospitals main system database to communicate wirelessly with the pump and determine the geographic location of each one of the medical pumps.

In order to periodically update the pumps in the field, a software uploading device may be programmed to wirelessly transmit software to each one of the medical pumps as well as updated drug libraries with current lists of drug information.

Real time monitoring devices located in each one of the medical pumps may be programmed to receive sensed information from sensors and wirelessly transmit the sensed information to the central database where it may be accessed by the hospital's main system database. The sensed information may include, but is not limited to, blood pressure, pulse, temperature, heart rhythm or any other diagnostic information.

A compliance device in each one of the pumps may be programmed to monitor the pump's operation and verify orders are successfully executed. Reports may then be generated by the compliance device and wirelessly transmitted a message listener on the central server that may also be accessed by the hospital's main system database.

Any information gathered or stored on the central database may be made easily accessible to the hospital's main system database with the handshake and filtering device discussed above. The system not only collects and transmits medical device information but also provides a central management tool for all of an organizations medical devices. This centralization allows management of the population of medical devices and access to both the devices and their information in one location. Additionally, the procedures mentioned above with respect to the medical liquid pumps may be employed on any medical device. The invention allows for the procedure to also be retrofitted on any existing device that is manufactured by any company.

These and other aspects and objects of the present invention will be better appreciated and understood when considered in conjunction with the following description and the accompanying drawings. It should be understood, however, that the following description, while indicating preferred embodiments of the present invention, is given by way of illustration and not of limitation. Many changes and modifications may be made within the scope of the present invention without departing from the spirit thereof, and the invention includes all such modifications.

### BRIEF DESCRIPTION OF THE DRAWINGS

A clear conception of the advantages and features constituting the present invention, and of the construction and operation of typical mechanisms provided with the present invention, will become more readily apparent by referring to the exemplary, and therefore non-limiting, embodiments illustrated in the drawings accompanying and forming a part of this specification, wherein like reference numerals designate the same elements in the several views, and in which:

FIG. 1 is a schematic illustration of a device communication application which may be referred to as a vendor neutral enterprise system, or ViNES, according to an exemplary embodiment;

FIG. 2 is an illustration of a device communicator and processor of FIG. 1, configured to facilitate device communication with a large number of possible devices and communications engines, is shown according to an exemplary embodiment;

FIG. 3 is a depiction of an exemplary ViNES object, a computer implemented record stored in non-transitory memory of the system of FIG. 1, according to an exemplary embodiment;

FIG. 4 is a flowchart illustrating how the system of FIG. 1 integrates with hospital infrastructure, according to an exemplary embodiment; and

FIG. 5 illustrates an interface for interacting with and controlling the operation of the system of FIG. 1, according to an exemplary embodiment.

In describing the preferred embodiment of the invention which is illustrated in the drawings, specific terminology will be resorted to for the sake of clarity. However, it is not intended that the invention be limited to the specific terms so selected and it is to be understood that each specific term includes all technical equivalents which operate in a similar manner to accomplish a similar purpose. For example, the words “connected”, “attached”, or terms similar thereto are often used. They are not limited to direct connection but include connection through other elements where such connection is recognized as being equivalent by those skilled in the art.

### DETAILED DESCRIPTION OF EMBODIMENTS

The present invention and the various features and advantageous details thereof are explained more fully with reference to the non-limiting embodiments described in detail in the following description.

### 1. System Overview

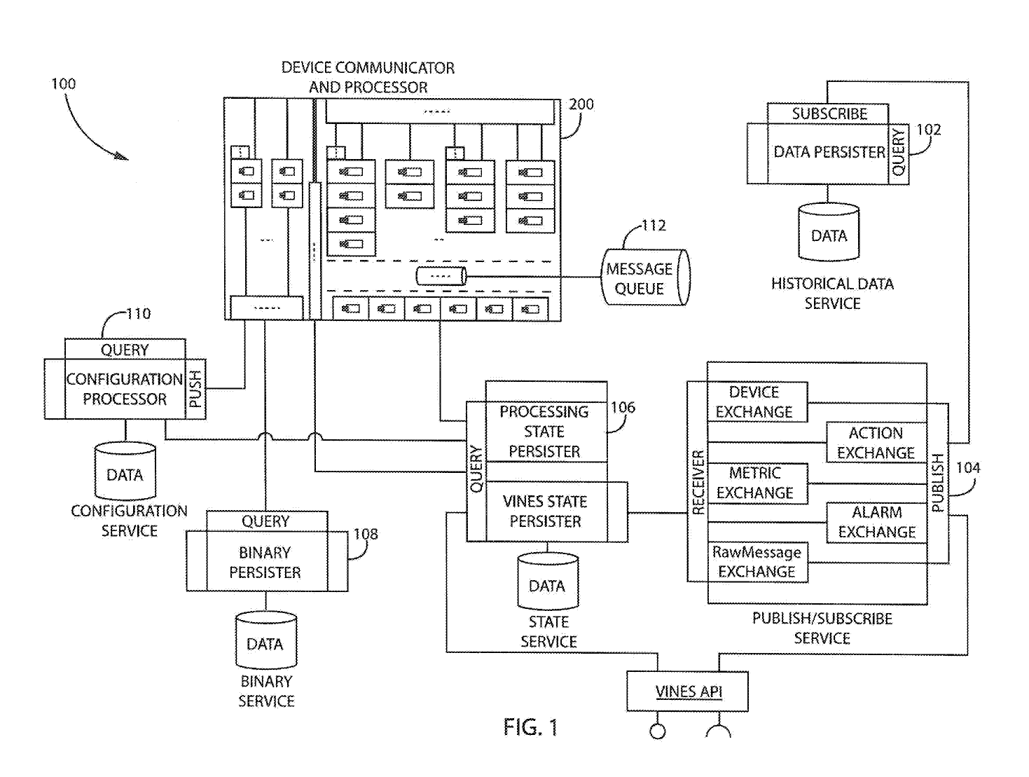

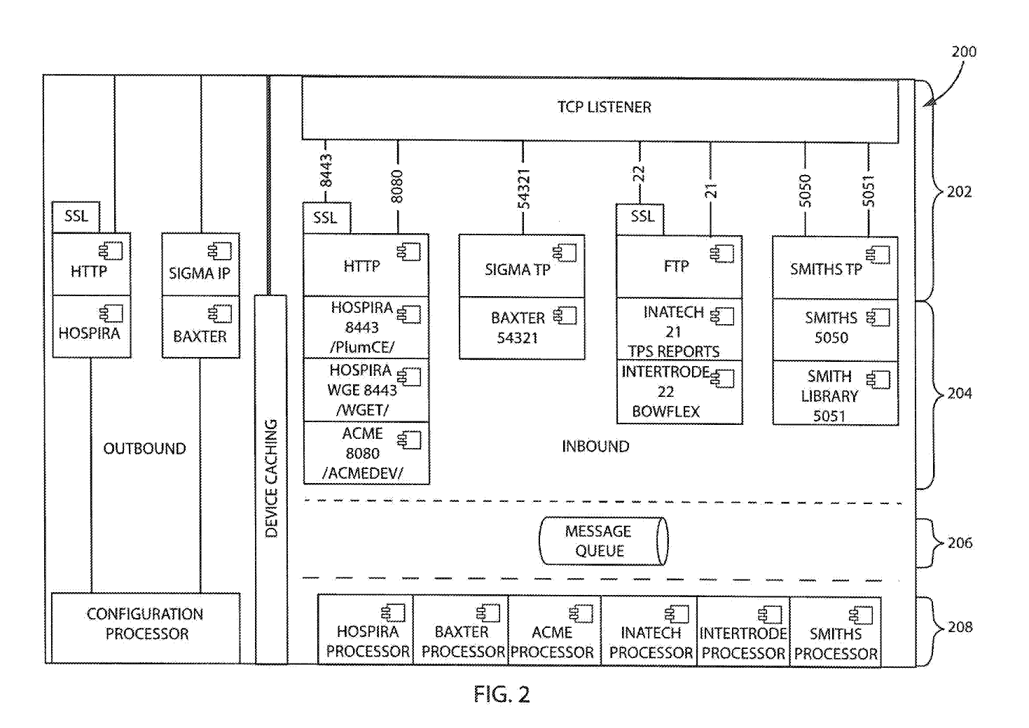

Referring now to FIG. 1, one example of the invention includes a device communication application 100 which may be referred to as a vendor neutral enterprise system, or ViNES, according to an exemplary embodiment. The core functionality of ViNES will allow deployed medical devices to connect to and communication engine to connect from, a set of installable modules could allow common functions such as: device management, data reporting, integration support, and real-time monitoring. Accordingly, application 100 include a device communicator and processor 200, described in further detail below with reference to FIG. 2, a historical data service 102, a publish and subscribe service 104, a state service 106, a binary service 108, and a configuration service 110.

Application 100 may be implemented using one or more standard computer systems including a computer processor, memory, and input and output devices. The memory can include both short term random access memory (RAM) and long term storage, such as a disk drive. Input and output device can include keyboards, monitor, data connections such as the Internet, etc.

ViNES application 100 may be designed to accommodate not only medical infusion pumps, but a broad spectrum of medical devices which may include respirators, patient monitors, and more. Additionally, the invention may not be limited to a single device manufacture, but may be employed to a broad range of devices by different manufacturers.

Referring now also to FIG. 2, device communicator and processor 200, configured to facilitate device communication with a large number of possible devices and communications engines, is shown according to an exemplary embodiment. For example, different devices from different manufacturers may all communicate on the same IP address and/or port. This is accomplished through custom device operating systems, firmware, operating libraries, etc. which can all be handled differently on different devices. Different devices may employ unique protocols and/or handshakes and therefore are all completely different.

It is the inventions objective to allow all these different devices including modular solution built around a common core engine. Device communicator and processor 200 may be implemented as a computer software application including instructions performed by the processor described above with reference to FIG. 1. Device communicator and processor 200 may be a single service that can be installed on several servers in a high availability, fault tolerant configuration. The core engine may be licensed as part of the standalone products or compile directly into a solution for a customer specific device.

The invention may include a native protocol that device manufacturers could adapt based on their type of device. Each module may include a reporting engine and an integration engine. The module may support multiple devices such as infusers, respirators, monitors, etc. as well as multiple transports. The device communicator and processor 200 includes a plug-in architecture for implementing transfer protocols 202, preprocessors 204, message queuing and durable storage 206, and device specific processing modules 208. These modules can be loaded at service start up and inserted/removed during runtime.

Preprocessors 204 will register with a transfer protocol 202 by sending it a port, using a secure socket layer (SSL) flag, using an option IP address, and providing a series of bytes that can be used as a signature to identify the message type. This data will be used by the transfer protocols 202 to route the message to the correct preprocessor 204. Transfer protocols 202 will listen for data on all the preprocessor registered ports and try to match the signature data that is registered for each individual port. Transfer protocol 202 will then send the data to the appropriate preprocessor 204. If only one preprocessor 204 registers for one port in the transfer protocol 202, no matching will occur and the data will be passed directly from the transfer protocol 202 to the preprocessor 204.

Preprocessors 204 are configured to read the message data to find a device identifier and compared to a list of known devices. The devices known to unauthorized, or if the device is unknown, it will reject the message and send an unauthorized device message through the system messaging to 112, shown above with reference to FIG. 1, so that another system can potentially use the messages. If the preprocessor 204 is not the owner of that device, preprocessor 204 is configured to query the state service 106 for the name of an alternative device communicator and processor 200 that is the owner of the device and notify that system to give up control. Preprocessor 204 will then signal the new device processor 200 to start processing for that device.

Preprocessors 204 are configured to store authorize message in the message queue for that device in the message queue durable storage 206. During message storage and formulation, preprocessors 204 will provide functions such as duplicate message detection.

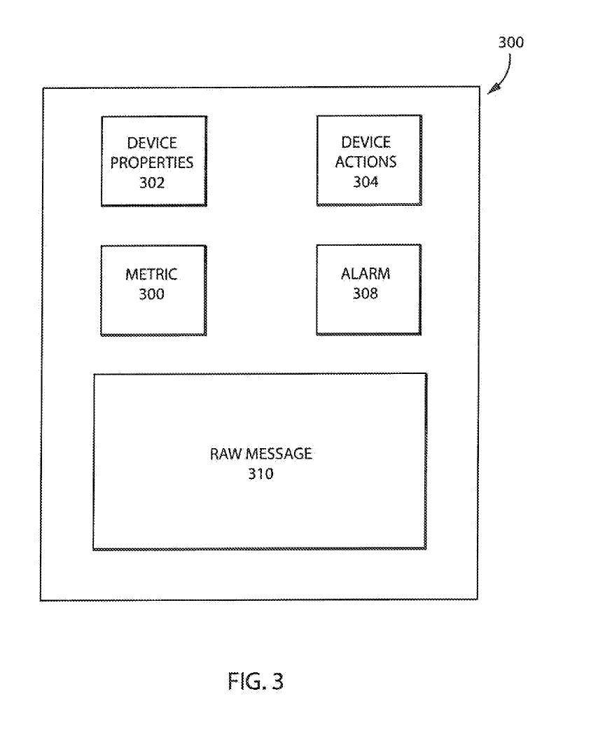

Device processors 208 are configured to pull messages out of the messaging queues 206 and process the messages in order to translate them into a ViNES specific device, properties, action, metric, or alert object, further described below with reference to FIG. 3. Device processors 208 are further configured to store a processing state object to the state service 106 in case the processor system 200 fails and the processing needs to occur on another processor 200. Once a device processor 208 has enough information to translate messages to a ViNES object or property, it will save that ViNES object or property to the state service 106. The device processor 208 will be able to set “do not save state”, “nonpersistent,” and “non-transactional” flags on a message in order to bypass durable storage and guaranteed delivery processes. In the simplest configuration, a device processor 208 may be configured to send a device's raw messages as a RawMessage object.

Referring again to FIG. 1, state service 106 is a computer application configured to store and provide access to objects that are used by various services of the system 100. As processing of messages occurs, properties are saved to a central state service 106 such that, in the case of a DCP 200 failure, another DCP 200 can take over from where the last one left off. Accordingly, the state service 106 will transmit the state of a device and is objects in response to a query from any other service of the system 100. State service 106 will store processing state objects and ViNES objects for all device processors 208.

State service 106 is further configured to inject messages into the publish and subscribe service 104 based on a detected change to the ViNES object. Once a device processor 208 has determined that no more updates will occur to the top-level object, the device processor 208 will mark as complete, and the state service 106 will then send a final object complete message using publish and subscribe service 104 and delete that object. State service 106 will send a message to the publish and subscribe service 104 if the “non-transaction” flag is set in the ViNES object and will bypass the guaranteed message delivery process and make transmitting the data faster. Similarly, state service 106 will mark a message as nonpersistent if the “nonpersistent” flag is set. State service 106 will further transmit a message to the publish and subscribe system 104 constructing the system to not save the message to disk. Although this can lead to data loss if the system were to be stopped while message was only in temporary memory, this would make transmission of the data faster.

Publish and subscribe service 104 is a computer application configured to send out state change messages to interested systems. Publish and subscribe service 106 allows interested systems to subscribe to a set of object properties in order to receive data from objects that match those properties. The properties that can be subscribed include at least device ID object type, group ID, and transaction ID. System 100 will allow for different devices to store different data in a system 200 database (not shown). According to an exemplary embodiment, data is stored in historical data service 102 using key value pairs, and the definitions for this data are included in the plugins in processors 208. Each device may have a specific ‘template’ in the database. For instance, all infusion devices would have a standard template for the data. There can be a method or unique file format that allows for industry-standard data (such as standards being developed by IEEE) but system 200 may also allow for device or manufacturer specific data.

Accordingly, data from different devices is stored in the database in a common format, allowing systems to ‘subscribe’ to the data based off of a certain parameter—patient name, patient ID, room number, bed number, hospital floor, unit, specific hospital, etc. System 200 amalgamates data from a larger number of disparate types and manufacturers for devices into a meaningful report or analytic based off a common identifier.

For example, a caregiver may like to see the infusion, heart rate, temperature and oxygen saturation readings for a specific patient for the past 24 hours. Accordingly, the caregiver is able to subscribe to a set of linked datapoints that provide a replay of a patient's condition over long (+3 days) period of time without having to log into each unique system for the devices, find the data and then compile all of it manually. Today, the majority of this data is either lost or stored in disparate databases and systems, making it all but impossible to retrieve without significant effort in time and resources. System 200 is designed to simplify this process.

Publish and subscribe service 104 further allows a system to request the current state of all objects currently subscribed to that system even in advance of receiving state changes messages for those objects. A subscriber can further create a persistent message queue which would allow the accumulation of messages even if the system that is subscribing is off-line. A subscriber may further be able to re-create a ViNES object using the state change updates of that object supplied by the state service 106.

Configuration service 110 is a computer limited application configured to provide an asynchronous device update, such as updating a drug library stored on a device, as well as perform additional processing and tracking functions described below. Configuration service 110 is configured to receive configuration requests from other services of the system 100 and order to send data to a device. The configuration requests may include any type of configuration from “set screen color” to “upload firmware.” Configuration 110 is configured to request and confirm successful updating configuration information by the device. Configuration service 110 is further configured to transmit queries to state service 106 to determine which DCP 200 is currently responsible for device and thereafter send that DCP 200, the configuration request. Thereafter, the DCP 200 and will respond the configuration service 110 with status information for the configuration request.

Binary service 108 is a computer implemented application configured to hold large binary objects that can be referenced within system messages, such as the aforementioned drug library file, firmware files, etc. Objects like an infuser drug library file will be loaded into the binary service 108. When a service needs access to this binary, for example during a drug library update, described above with reference to configuration service 110, the service was supply a binary ID and received the binary object. Binary service 108 may further be utilized if a device sends large data such as images or sound files.

Historical data service 102 is a computer implement application configured to store historical data provided by the state service 106. Historical data service 102 can be configured to allow subscription to any combination of object properties and receive state updates from the publish and subscribe service 104. Thereafter, service 102 will then store the updates into a system database (not shown) such other services can access the historical object database.

Referring now to FIG. 3, an exemplary ViNES object 300, is a computer implemented record stored in non-transitory memory of the system 100, according to an exemplary embodiment. Object 300 is configured to include a device properties record 302, device actions record 304, a metric record 306, an alarm record 308, and a raw message 310 data field. Device properties record 302 is configured to hold the properties of device such as, but not limited to, IP address, serial number, etc. The device actions record 304 is configured to include a listing of objects that represent actions that may be taken by a device associated with records such as, but not limited to, infusion, ventilation, etc. Metric record 306 holds the property of a patient measurement. Alarm record 308 holds objects that represent alarms generated by a device. Raw message data field 310 is configured to include a raw message from a device.

Referring again to FIG. 1, the information that each module in DCP 200 outputs may be received by one or more of an integration engine, a reporting database, a custom dashboard, etc. the integration should support various protocols including IHE PCD01 for messages, IHE PCD02 for subscription, IHE PCD03 for auto programming features, IHE PCD09 for user definable alarms, the ability to connect to existing software such as Cerner and Epic, and support universal medical device communication standard. If no universal medical device communication standard exists, the invention may be used as a model for such a standard.

A website may allow users to login and access the stored information as well as manage the devices. There may be a single site at a hospital that could work for any device regardless of the particular brand or type of device. Reports may then be generated that each device that are accessible through the website.

The interface that allows users to use the invention may include a web based interface that is accessible through the Internet. The interface may also be able to accept branding or skins that allow certain manufacturers to add desired looks or visual appearances to the interface.

Security measures may also be employed to the interface such as internal forms based security and LDAP-based security. Full enterprise-wide organization, region, group, facility, users, and role architecture may also be employed in order to establish predetermined hierarchies for potential users. The interface may also allow for localization or internationalization allowing it to be used in any particular country or region. Interface may also be adaptable allowing for mobile phone or smart phone compatible display. Stored data may also be filtered so that the data that is displayed may be tailored to make more sense to the user. The interface may also include device configuration and auto configuration utilities that allow it to be set up and administer with minimal setup time and input from the user. The interface may also allow files to be distributed, also known as pushed, to the various devices which include operating system updates, firmware updates, drug library updates, configuration files, etc.

### 2. Detailed Description of Preferred Embodiments

The present invention and the various features and advantageous details thereof are explained more fully with reference to the non-limiting embodiments described in detail in the following description.

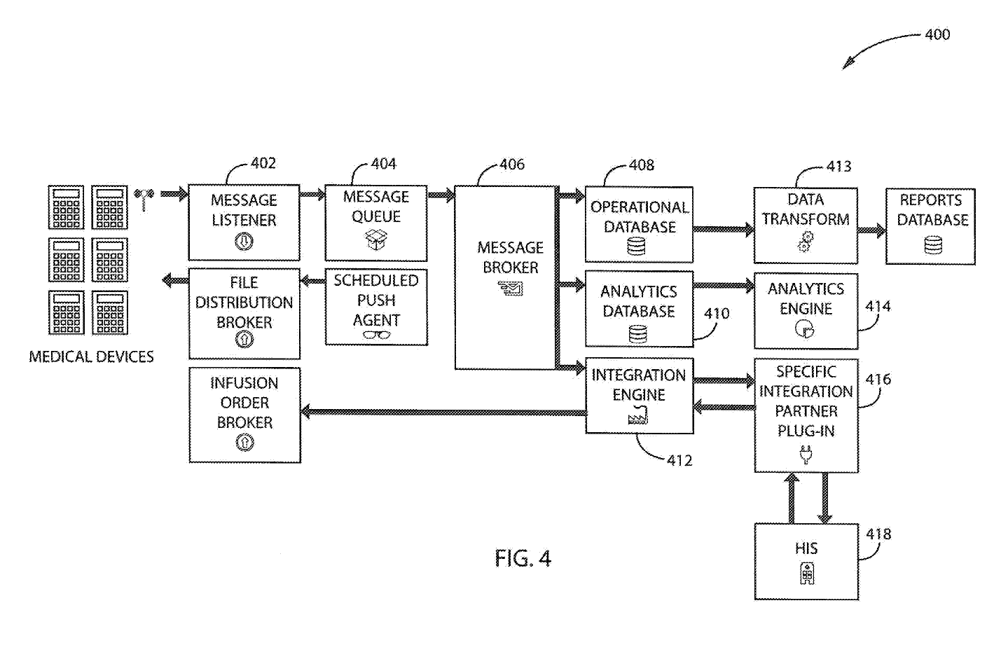

In accordance with the invention, FIG. 4 is a flowchart illustrating a computer implemented method for how medical devices wirelessly transmit information to a message listener. These methods may be implemented by one or more of the systems shown in FIG. 1 in combination with external devices and systems.

Referring first to FIG. 4, method 400 includes a message listener may include software loaded on a computer that is programmed to receive information in the form of a message regardless of the source in a step 402. A few examples of a medical device that may send a message to the message listener include, but are not limited to, an MRI machine, an x-ray machine, a medical liquid pump, and ultrasound machine, a heart rate monitor, a telephone, a fax machine or any other device that is used in the medical field.

After receiving a message the message listener may store the messages in the message queue in a step 404. A message broker may be programmed to distribute messages to multiple locations in a step 406. These multiple locations may include but are not limited to a operational system database 408, an analytics database 410, and integration engine 412. The main system database may perform ETL on the received messages in order to generate reports that are stored in a report database in a step 413. The analytics engine 414 may process and display real-time analytics from the data stored in the analytics database. The integration engine 416 may be coupled with a specific integration partner plug-in in order for the data to be accessible and used by the main hospital information system 418. The main hospital information system may include but is not limited to Cerner, Epic, McKesson or other similar programs.

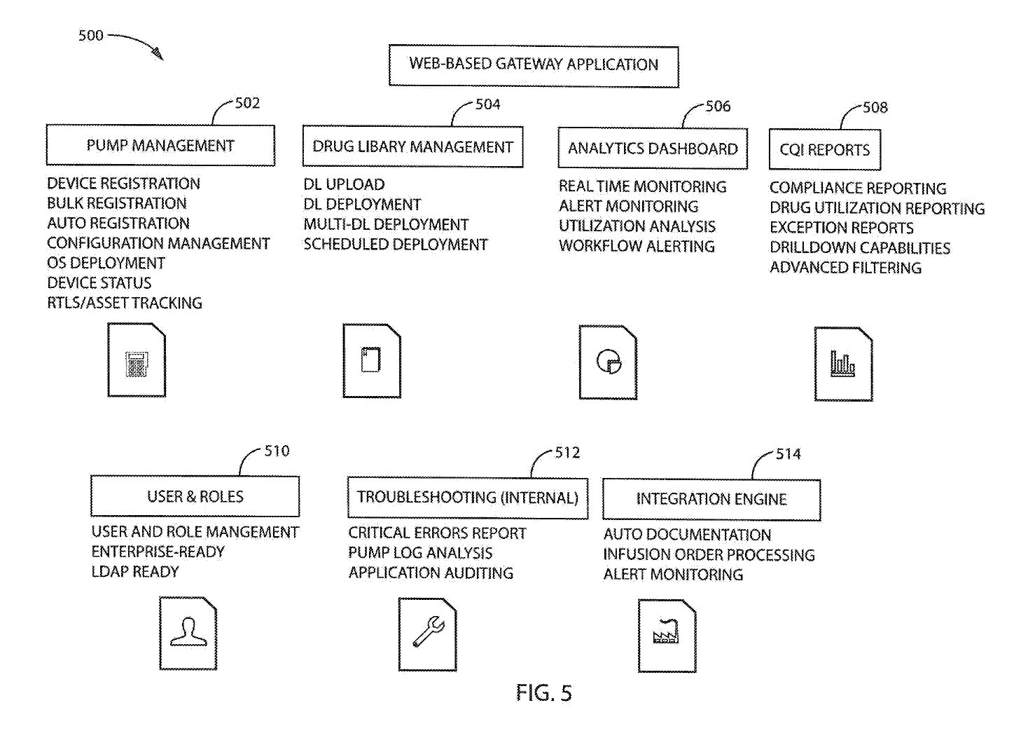

Referring now to FIG. 5, a block diagram is provided that describes a computer implemented gateway application 500 describing how the main hospital information system may then communicate with the specific partner plug in for generating orders for the medical devices. The integration engine passes these orders to an infusion broker, when an infusion pump is the device, and the push agent queues files to be distributed by the file distribution broker. For example, in one embodiment the medical device may include the medical liquid pumps for the administration of liquid drugs to patients. The system may be designed to integrate any manufacturer's brand of pump to ambulatory and hospital information systems. The system may be generic and allow connectivity to any manufacturer's pump.

Referring now to FIG. 5, a web based Gateway application 500 may be used so the system may manage all the pumps in an organization including the pumps located in ambulances and hospital rooms. The following features may therefore be accessible at any time with internet access to the central database, or cloud.

Pump management 502 may be utilized to auto register, bulk register, keep track of the various pumps in the field including configuration management, operating system deployment, device status, and asset tracking. The web based Gateway application may also push operating system updates automatically and wirelessly to each pump. The message broker may be used to wirelessly push an infusion order to the pump notifying the medical personnel to administer a drug to a patient. The pushed messages may also include drug library management information that includes an entire library of drug information 504. The drug library may be periodically updated through scheduled deployments by the system and may be uploaded or downloaded to each device.

The system may also wirelessly monitor each pumps operation in the field to assist in the hospital in tracking the utilization of every pump in the field using CQI reports 508 as well as allow for real-time monitoring in the hospital by any authorized personnel identified by users & roles database display 510. The pumps may be further programmed to wirelessly transmit alerts to the system in order to notify qualified hospital personnel that an infusion order was properly carried out, was failed to be carried out, or that the patient's vitals reached a predetermined threshold. Workflow may also be monitored and alerted though an analytics dashboard application 506.

An added feature may include programming every pump in the field to calculate quality reports detailing the drug utilization, drilldown capabilities, advanced filtering, and compliance with any predetermined requirements, expectation reporting, or any other benchmark the hospital chooses to monitor.

This system may also wirelessly update each pump in the field to allow certain users access to operate the pump. Different users may be assigned different authorizations in database 510. The system may also be LDAP ready and enterprise ready allowing seamless integration with existing hospital IT infrastructure.

The system may be updated, repaired, or serviced wirelessly by an authorized party using troubleshooting interface 512. This allows for remote third-party troubleshooting and auditing should any problems arise in the field.

The integration engine 514 may also allow for auto documentation regarding order processing and alert monitoring.

Although the best mode contemplated by the inventors of carrying out the present invention is disclosed above, practice of the present invention is not limited thereto. It will be manifest that various additions, modifications and rearrangements of the features of the present invention may be made without deviating from the spirit and scope of the underlying inventive concept. Furthermore, all the disclosed features of each disclosed embodiment can be combined with, or substituted for, the disclosed features of every other disclosed embodiment except where such features are mutually exclusive. It is intended that the appended claims cover all such additions, modifications and rearrangements. Expedient embodiments of the present invention are differentiated by the appended claims.

What is claimed is:

1. A medical liquid pump system comprising:
a plurality of medical pumps;
a message listener programmed to wirelessly receive a plurality of messages from the plurality of medical pumps;
a storage device in communication with the message listener for storing the messages;
a message distribution device for delivering the messages to a plurality of locations including a system database, an analytics database, and an integration engine wherein:
1) the system database performs extract, transform, and load “ETL” operations on the messages and generates reports;
2) the analytics database computes and displays a real time analytic information;
3) the integration engine is programmed to transmit the messages and receive a plurality of feedback from a central information system;
the main information system is programmed to produce an order for a pump in the plurality of medical pumps; and
the pump is configured to wirelessly receive and display the order.

2. The medical liquid pump system of claim 1, further including:
a drug library with a plurality of drugs identified in the drug library programmed into each one of the plurality of medical pumps wherein; and
the pump is programmed to identify the order and associate a drug from the order with a drug from the drug library.

3. The medical liquid pump system of claim 2, further including a locator installed in each one of the plurality of medical pumps wherein the central information system communicates wirelessly and is programmed to display a geographic location of each one of the plurality of medical pumps.

4. The medical liquid pump system of claim 3, further including a software uploading device programmed to wirelessly transmit:
1) software to each one of the plurality of medical pumps; and
2) a drug library update for updating the plurality of medical pumps with a current list of drug information.

5. The medical liquid pump system of claim 4, further including a real time monitoring device located in each one of the plurality of medical pumps programmed to receive sensed information from at least one sensor wherein the sensed information includes at least one of blood pressure, pulse, temperature, and heart rhythm.

6. The medical liquid pump system of claim 5, further including a compliance device in each one of the plurality of medical pumps programmed to monitor a pump operation to verify the order was successfully executed, and wherein a report is generated by the compliance device and wirelessly transmitted to the message listener.

7. A medical information management system comprising:
a central database programmed to receive a data from a plurality of medical devices;
a plurality of input devices programmed to wirelessly transmit the data to the central database;
a handshake device programmed in the central database to translate all received data into a language readable by the central database and by a plurality of output devices in wireless communication with the central database;
wherein the input devices includes medical diagnostic equipment, medical pumps, medical imaging devices, and any device capable of gathering an information of a hospital patient.

8. The medical information management system of claim 7, further including a module located within each one of the input devices programmed to wirelessly transmit the data to the central database.

9. The medical information management system of claim 8, further including a filtering device in communication with the central database and programmed to filter the data into a group of distinguished categories.

10. The medical information management system of claim 9, wherein output devices include least one or more of a personal computer, a smart phone, and a tablet computer.

11. The medical information management system of claim 10, wherein the central database is located on a cloud server which is accessible wirelessly in a location where a wireless cell phone signal is reachable.

12. A medical device communication system comprising:
a plurality of medical devices;
a message listener programmed to wirelessly receive a plurality of messages from the plurality of medical devices;
a storage device in communication with the message listener for storing the messages;
a message distribution device for delivering the messages to a plurality of locations including a system database, an analytics database, and
an integration engine, wherein
the system database performs extract, transform, and load “ETL” operations on the messages to generate system specific message objects, the system specific message objects configured to include the received message used to generate the system specific message object,
the analytics database computes and displays a real time analytic information stored in each system specific message objects, and
the integration engine is programmed to transmit the system specific message objects and receive a plurality of feedback from a central information system.

13. The medical device communication system of claim 12, wherein the medical device is a medical pump, further including:
a drug library with a plurality of drugs identified in the drug library programmed into each one of the plurality of medical pumps wherein; and
the pump is programmed to identify the order and associate a drug from the order with a drug from the drug library.

14. The medical device communication system of claim 12, further including a locator installed in each one of the plurality of medical devices wherein the central information system communicates wirelessly and is programmed to display a geographic location of each one of the plurality of medical devices.

15. The medical device communication system of claim 14, further including a software uploading device programmed to wirelessly transmit software to each one of the plurality of medical devices; and a drug library update for updating the plurality of medical devices with update information including at least one of firmware and medical information.

16. The medical device communication system of claim 15, further including a real time monitoring device located in each one of the plurality of medical devices programmed to receive sensed information from at least one sensor wherein the sensed information includes at least one of blood pressure, pulse, temperature, and heart rhythm.

17. The medical device communication system of claim 16, further including a compliance device in each one of the plurality of medical devices programmed to monitor a devices operation to verify an order was successfully executed, and wherein a report is generated by the compliance device and wirelessly transmitted to the message listener.
